(19) United States
(12) Patent Application Publication
(10) Pub. No.: US 2018/0187130 A1
(43) Pub. Date: Jul. 5, 2018

(54) Granulates, Their Use and Washing Agent and Cleaning Agents Containing Same

(71) Applicant: WeylChem Wiesbaden GmbH, Wiesbaden (DE)

(72) Inventors: Paula Barreleiro, Ginsheim (DE); Roman Morschhäuser, Mainz (DE); Bo Kuhse, Mainz-Kastel (DE); Rolf Ludwig, Eppstein (DE)

(21) Appl. No.: 15/849,292

(22) Filed: Dec. 20, 2017

(30) Foreign Application Priority Data
Dec. 31, 2016 (DE) 102016015660.0

(51) Int. Cl. C11D 3/34; C11D 3/30; C11D 3/20; C11D 3/37; C11D 3/39; C07C 251/30; C11D 17/06

(52) U.S. Cl. CPC C11D 3/3454; C11D 3/349; C11D 3/30; C11D 3/2082; C11D 17/06; C11D 3/3707; C11D 3/3917; C07C 251/30; C11D 3/2086

(57) ABSTRACT

Disclosed are granulates comprising
a) one or more sulfonimines and/or one or more quaternary iminium salts, and
b) one or more acids which are solid at 25° C.

These granulates contain highly-active bleach catalysts and are very storage stable. The granulates show no tendency towards staining during storage and experience no appreciable loss of active substance during storage.

The granulates of the invention can preferably be used in dishwasher agents or in washing agents and cleaning agents for low temperatures.

### CLAIM FOR PRIORITY

This application is based on German Patent Application No. 10 2016 015 660.0, filed Dec. 31, 2016, the priority of which is hereby claimed and the disclosure of which is incorporated herein by reference.

### TECHNICAL FIELD

The present invention relates to granulates as well as to their use in washing agents and in cleaning agents, preferably in dishwashing agents.

### BACKGROUND

Dishwasher detergents incorporate persalts such as perborates and percarbonates for spotless results. To activate these bleaching agents and in order to achieve an improved bleaching effect at wash cycle temperatures of 60° C. or below, dishwasher detergents generally further contain bleach activators or bleach catalysts, and it is particularly the bleach catalysts which have proven to be particularly effective.

Bleach catalysts are preferably used in dishwasher detergents in the form of ready-made granules. This is done not only to increase their storage stability but also to ensure homogeneous incorporation of minuscule catalyst quantities into the formulations, in particular in tablet applications.

Sulfonimines and quaternary iminium salts have been described as bleaching catalysts in the patent literature already.

DE 691 04 405 T2 corresponding to EP 0 453 003 B1 discloses bleaching compositions for washing agents comprising 1 to 60% by weight of a peroxygen compound, 0.05 to 10% by weight of a selected sulfonimine and 0.5 to 50% by weight of a surfactant.

DE 691 04 751 T2 corresponding to EP 0 446 982 B1 discloses bleaching compositions comprising 1 to 60% by weight of a peroxygen compound, 0.05 to 10% by weight of a selected sulfonimine and 0.1 to 40% by weight of a bleach percursor reacting with the peroxide anion.

DE 696 14 224 T2 corresponding to EP 0 775 192 B1 discloses bleaching compositions comprising 1 to 60% by weight of a selected peroxygen compound, 0.01 to 10% by weight of a C₁-C₃₀-imine and 0.001 to 40% by weight of a selected transition metal catalyst.

DE 694 05 407 T2 corresponding to EP 0 728 182 B1 discloses bleaching compositions comprising 1 to 60% by weight of a peroxygen compound, 0.01 to 10% by weight of a selected quaternary iminium salt as an oxygen transfer agent and 0.5 to 50% by weight of a surfactant.

DE 694 09 252 T2 corresponding to EP 0 728 183 B1 discloses bleaching compositions comprising 1 to 60% by weight of a peroxygen compound, 0.01 to 10% by weight of a selected quaternary iminium salt as an oxygen transfer agent and 0.1 to 40% by weight of a bleaching agent precursor reacting with a peroxide anion.

Granulates comprising bleach catalysts are known. WO 03/093405 discloses granulates comprising bleach catalyst, bleach activator and optionally a coating. Preferred bleach catalysts for preparing such granulates are metal complexes with macrocyclic bridged ligands, such as disclosed in WO 01/64826 and WO 98/39098. WO 2010/115581 discloses granulates comprising bleach activators, bleach catalysts and at least 5% by weight of one or more organic acids. Transition metal salts or complexes of manganese, iron, cobalt, ruthenium, molybdenium, titanium or vanadium are mentioned as possible bleach catalysts.

G. Reinhardt, M. Best, I. Herrgen and M. Ladwig disclose in SOFW-Journal 140 9-2014 sulfonimines and quaternary iminium salts as novel bleach catalysts. These compounds serve as precursors of dioxiranes, oxaziridines and oxaziridinium salts, which show already in low concentration in combination with per-compounds an excellent bleaching action. These bleach catalysts are applied in basic milieu in which they develop their highest effect. It has been proven that sulfonimines and quaternary iminium salts tend towards decomposition during storage in solid form. Apart from loss of active substance going along with this undesirable discoloration occurs. In the publication mentioned above it has been already proposed to use sulfonimines and quaternary iminium salts in the form of granulates together with additional bleach activators, such as tetraacetylethylene diamine (TAED) or nonanoyloxibenzene-sulfonate-sodium (NOBS) and to use also film-forming binders. But those granulates often are not sufficiently storage-stable, especially when components comprising small amounts of water are used.

### SUMMARY OF INVENTION

Surprisingly, it was also found that bleaching agents in combination with sulfonimines or quaternary iminium salts together with other bleach activators are suitable for the formulation of so-called detergents that work at low temperatures. These combinations allow an excellent bleaching effect already at low temperatures of e.g. of less than 35° C. In addition to the bleaching effect, these combinations have a pronounced tendency to destroy micro-organisms.

Starting from the prior art, the objective of the present invention is the provision of granulates which are storage-stable for a prolonged time, which contain highly active bleach catalysts, which do not tend to discoloration during storage and which do not suffer from appreciable loss of active substance during storage.

Another objective of the present invention is the provision of granulates, which in combination with bleaching agents allow excellent washing results at low temperatures and which drastically reduce the number of micro-organisms on the treated substrates.

These objectives are solved by provision of granulates comprising
a) one or more sulfonimines and/or one or more quaternary iminium salts, and
b) one or more acids which are solid at 25° C.

The sulfonimines used according to the invention are compounds having one or two structural units ═N—SO₂— in the molecule.

The quaternary iminium salts used according to the invention are compounds having one or two structural units >C═N⁺— in the molecule.

Preferably used sulfonimines are compounds of formula I

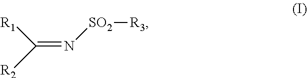

in which R₁ is hydrogen, alkyl, cycloalkyl, aryl, aralkyl or heterocyclyl, wherein the residues alkyl, cycloalkyl, aryl, aralkyl or heterocyclyl are unsubstituted or carry one to three substituents selected from the group alkyl, alkoxy, aryl, heterocyclyl, amino, carboxyl, carboxylic ester, carboxylic amide, sulfonyl, cyano, hydroxy or halogen, or wherein an alkyl group is interrupted by one or more oxygen atoms which are not directly adjacent or by one ore more keto groups which are not directly adjacent,
R₂ is hydrogen, alkyl, alkoxy, cycloalkyl, aryl, aralkyl, heterocyclyl, nitro, cyano, halogen, carboxyl, carboxylic ester or carboxylic amide, in which the residues alkyl, cycloalkyl, aryl, aralkyl or heterocyclyl are unsubstituted or have one to three substituents selected from the group alkyl, alkoxy, aryl, heterocyclyl, amino, hydroxy, carboxyl, carboxylic ester, carboxylic amide, sulfonyl, cyano or halogen, or in which the residues alkyl, cycloalkyl, aryl or heterocyclyl are substituted with a residue of formula —R₁C═N—SO₂—R₃ or in which an alkyl group is interrupted by one or more not directly adjacent oxygen atoms or by one ore more keto groups which are not directly adjacent,
R₃ is alkyl, cycloalkyl, aryl, aralkyl, heterocyclyl, nitro, cyano or halogen, in which the residues alkyl, cycloalkyl, aryl, aralkyl or heterocyclyl are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one ore more keto groups which are not directly adjacent, or in which
R₁ and R₂ together with the carbon atom to which they are attached form a cycloaliphatic, heterocyclic or carbocyclic-aromatic ring system, which are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one ore more keto groups which are not directly adjacent, or in which
R₂ and R₃ together with the group —C═N—SO₂—, via which they are connected, form a heterocyclic ring system which is unsubstituiert or has one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one ore more keto groups which are not directly adjacent.

Especially preferred used sulfonimines are compounds of formula I, wherein R₁ is hydrogen, R₂ is an unsubstituted phenyl or a phenyl which is substituted in 4-position with carboxyl, chlorine, cyano, hydroxy or methoxy, and R₃ is unsubstituted phenyl or phenyl which is substituted in 4-position with carboxyl, chlorine or nitro.

Most preferably used sulfonimines are compounds of formula II

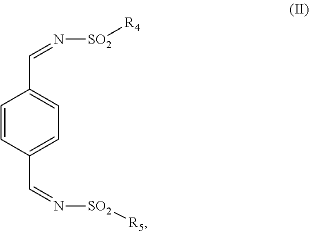

in which R₄ and R₅ independently of one another are unsubstituierted phenyl or phenyl that has been substituted in 4-position with carboxyl, chlorine, cyano, hydroxy or methoxy.

Further sulfonimines which are especially preferably used are compounds of formula Ill

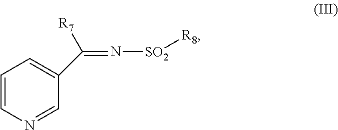

in which R₇ is hydrogen, C₁-C₆-alkyl or phenyl, and
R₆ is unsubstituted phenyl or phenyl which has been substituted in 4-position with carboxyl, chlorine, cyano, hydroxy or methoxy.

Most preferably used sulfonimines are compounds of formula IV

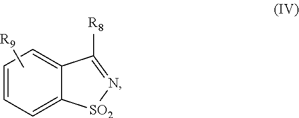

in which R₈ and R₉ independently of one another are hydrogen, C₁-C₆-alkyl or phenyl.

From the compounds of formula IV those are especially preferred, in which R₉ is hydrogen and R₅ is C₁-C₆-alkyl, preferably methyl or ethyl, or phenyl.

Most preferably used sulfonimine of formula IV is 3-methyl-1,2-benzisothiazole-1,1-dioxide.

Further sulfonimines which are preferably used are compounds of formula V

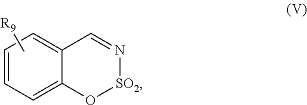

in which R₉ is hydrogen, C₁-C₆-alkyl or phenyl, preferably hydrogen.

Preferably used quaternary iminium salts are compounds of formula VI

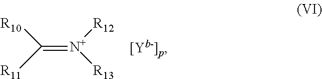

in which R₁₀ and R₁₂ independently of one another are hydrogen, alkyl, cycloalkyl, aryl, aralkyl or heterocyclyl, in which the residues alkyl, cycloalkyl, aryl, aralkyl or heterocyclyl are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more more not directly adjacent oxygen atoms or by one ore more not directly adjacent keto groups,
R₁₁ is hydrogen, alkyl, alkoxy, cycloalkyl, aryl, aralkyl, heterocyclyl, nitro, cyano, halogen, carboxyl, carboxylic ester or carboxylic amide, in which the residues alkyl, cycloalkyl, aryl, aralkyl or heterocyclyl are unsubstituted or have one to three substituents selected from the group alkyl, alkoxy, aryl, heterocyclyl, amino, hydroxy, carboxy, carboxylic ester, carboxylic amide, sulfonyl, cyano, hydroxy or halogen, or in which an alkyl group is interrupted by one or more not directly adjacent oxygen atoms or by one ore more not directly adjacent keto groups,
R₁₃ is alkyl, cycloalkyl, aryl, aralkyl, heterocyclyl, nitro, cyano or halogen, in which the residues alkyl, cycloalkyl, aryl, aralkyl or heterocyclyl are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one ore more not directly adjacent keto groups, or in which
R₁₀ and R₁₂ together with the group —C═N⁺—, over which they are attached to one another, form a heterocyclic ring system, which is unsubstituted or has one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more non directly adjacent oxygen atoms or by one ore more not directly adjacent keto groups, or in which
R₁₁ and R₁₃ together with the group —C═N⁺— to which they are attached to one another, form a heterocyclic ring system, which is unsubstituted or has one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one ore more not directly adjacent keto groups,
Y is a b-valent inorganic or organic counterion,
b is an integer of at least 1, preferably from 1 to 3, more preferably of 1 or 2 and most preferably of 1, and
p is a rational number with value of 1/b.

Especially preferably used quaternary iminium salts are compounds of formula VII

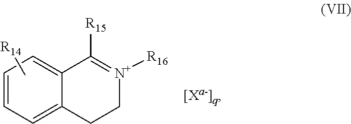

in which R₁₄ and R₁₅ independently of one another are hydrogen, alkyl, alkoxy, cycloalkyl, aryl, aralkyl, heterocyclyl, nitro, cyano, halogen, carboxyl, carboxylic ester or carboxylic amide, in which the residues alkyl, cycloalkyl, aryl, aralkyl or heterocyclyl are unsubstituted or have one to three substituents selected from the group alkyl, alkoxy, aryl, heterocyclyl, amino, hydroxy, carboxy, carboxylic ester, carbonxylic amide, sulfonyl, cyano, hydroxy or halogen, or in which an alkyl group is interrupted by one or more not directly adjacent oxygen atoms or by one ore more not directly adjacent keto groups,
R₁₆ is alkyl, aryl, aralkyl or heterocyclyl, in which these residues are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one ore more not directly adjacent keto groups,
X is an a-valent inorganic or organic anion, especially an anion which is selected from the group halogenide, preferably chloride or bromide, or tetrafluoroborate, hexafluorophosphate, hexafluoroantimonate, alkanesulfonate, most preferably methane sulfonate or ethane sulfonate, or aryl sulfonate, most preferably p-toluene sulfonate, and
a is at least 1, preferably 1 to 3, more preferably of 1 or 2 and most preferably 1, and
q is a rational number with value of 1/a.

Preferably a and q each are 1.

Very preferred quaternary iminium salts are used which are compounds of formula VII, in which

R₁₆ is C₁-C₁₅-alkyl, preferably methyl, ethyl, octyl, dodecyl or hexadecyl, or phenyl, benzyl, 2-hydroxyethyl, dimethylaminoethyl, ethoxyethyl or 2-pyridyl,
R₁₄ is hydrogen, C₁-C₁₅-alkyl, preferably methyl, ethyl or octyl, or trifluoromethyl, methoxy, nitro, fluorine, chlorine, bromine, cyano, phenyl, propyl-2-one, acetyl or pyridyl,
R₁₅ is hydrogen, C₁-C₆-alkyl, preferably methyl, or phenyl,
X is selected from the group chloride, bromide, tetrafluoroborate, hexafluorophosphate, methanesulfonate, ethanesulfonate or p-toluenesulfonate, and
a and q each are 1.

### DETAILED DESCRIPTION

If one of the groups referred to in this description stands for alkyl, the alkyl group can be branched or unbranched. An alkyl group typically contains one to twenty carbon atoms, preferably one to ten carbon atoms. Examples for alkyl groups are: methyl, ethyl, n-propyl, isopropyl, n-butyl, sec-butyl, tert.-butyl, pentyl, n-hexyl, n-heptyl, 2-ethylhexyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or eicosyl. Especially preferred are alkyl groups with one to six carbon atoms.

Alkyl groups can be optionally substituted, for example with alkyl, alkoxy, aryl, heterocyclyl, amino, hydroxy, carboxy, carboxylic ester, carboxylic amide, sulfonyl, cyano, hydroxy or halogen

Alkyl groups can be optionally interrupted by one or more oxygen atoms which are not directly adjacent. Examples thereof are monovalent residues derived from polyethylene glycols or from polypropylene glycols.

Alkyl groups also can be optionally interrupted by one or more keto groups which are not directly adjacent. One example thereof is the acetonyl group.

If one of the groups referred to in this description stands for cycloalkyl, the cycloalkyl group typically is a cyclic group comprising five to eight, preferably five, six or seven ring carbon atoms, which may be independently of one another substituted. Examples for substituents are mentioned in the antecedent disclosure of the alkyl group. Examples for cycloalkyl groups are cyclopentyl or cyclohexyl.

If one of the groups referred to in this description stands for aryl, the aryl group is typically a cyclic aromatic group comprising five to fourteen carbon atoms which independently of one another may optionally be substituted. Examples for substituents are mentioned in the antecedent disclosure of the alkyl group. Examples for aryl groups are phenyl, biphenyl or anthryl.

If one of the groups referred to in this description stands for heterocyclyl, the heterocyclyl group typically is a cyclic group with four to ten ring carbon atoms and with at least one ring heteroatom, which independently of one another may be optionally substituted. Examples for substituents are mentioned in the antecedent disclose of the alkyl group. Examples for heteroatoms are oxygen, nitrogen, phosphor, boron, selenium or sulfur. Examples for heterocyclyl groups are furyl, thienyl, pyrrolyl or imidazolyl. Heterocyclyl groups may be aromatic or non-aromatic.

If one of the groups referred to in this description stands for aralkyl, the aralkyl group typically is an aryl group, such as defined beforehand, to which an alkyl group is covalently attached. The aralkyl group may be substituted at the aromatic ring. Examples for substituents are mentioned in the antecedent disclosure of the alkyl group. An example for an aralkyl group is benzyl.

If one of the substituents referred to in this description stands for halogen, this is a fluorine-, chlorine-, bromine- or iodine-atom. Preferred are fluorine, chlorine or bromine.

The quaternary iminium salts used according to the invention, such as compounds of formulae VI or VII, contain counter ions, such as X^{a-} or Y^{b-}. These can be mono- or multivalent inorganic or mono- or multivalent organic anions or a mixture of such anions.

Examples of monovalent inorganic anions are halide ions, such as fluoride, chloride, bromide or iodide, or hydroxide ions or anions of inorganic acids, such as phosphate, sulfate, nitrate, borate, hexafluorophosphate, tetrafluoroborate, perchlorate, chlorate, hexafluoroantimonate, hexafluoroarsenate or cyanide.

Examples of multivalent inorganic anions are carbonate- or sulfate-anions.

Examples of organic anions are anions of mono- or polyvalent carboxylic acids or anions of mono- or polyvalent sulfonic acids, wherein these acids may by saturated or unsaturated. These can be, for example, anions of polycarboxylic acids or of polysulfonic acids. Examples for anions of polycarboxylic acids are polyacrylate- or polymethacrylate anions. An example for anions of polysulfonic acids is polystyrene sulfonate. Additional preferred examples for anions of organic acids are acetate, formiate, trifluoroacetate, methylsulfonate, ethylsulfonate, trifluoromethansulfonate, pentafluoroethanesulfonate, nonafluorobutanesulfonate, butyrate, citrate, fumarate, glutarate, lactate, malate, malonate, oxalate, pyruvate or tartrate.

In the granulate of the invention an acid solid at 25° C. is used as component b). This can be any inorganic or organic acid as long as this is solid at 25° C. Also mixtures of these acids can be used.

As inorganic acids all inorganic compounds in the form of free acid or as partial neutalisate may be used, as long as these are solid at 25° C. In the context of the present invention the term “inorganic acid” thus encompasses the inorganic acids in free form as well as in partial neutralized form.

Examples of inorganic acids are zeolites in H-form or partial neutralisated zeolites, which contain besided hydrogen ions still other ions, such as alkali-, earth alkaline- and/or transition metal ions.

As organic acids monomeric as well as polymeric acids can be used either in the form of the free acid or in partially neutralisated form, as long as these are solid at 25° C. In the context of the present invention the term “organic acid” thus encompasses the organic acids in free form as well as also in partially neutralized form.

Counter ions of organic acids are preferably alkali ions and more preferably sodium ions.

Preferred organic acids are citric acid, ascorbic acid, oxalic acid, adipic acid, sebacic acid, malonic acid, succinic acid, glutaric acid, malic acid, tartaric acid, lactic acid, maleic acid, fumaric acid, sugar acids, aminocarboxylic acids, C₈-C₂₂-fatty acids as well as mixtures from these. Especially preferred organic acids are oxalic acid, ascorbic acid, citric acid and C₈-C₂₂-fatty acids, for example lauric acid, myristic acid, palmitic acid or stearic acid.

As preferred polymeric organic acids polymers of acrylic acid or of methacrylic acid as well as copolymers of acrylic acid and/or of methacrylic acid with maleic acid may be used.

Some of the organic acids used as component b), such as fatty acids having 8 to 22 carbon atoms or as polymeric organic acids, in addition have the function of a binder.

Suitable fatty acids having 8 to 22 carbon atoms are, for example, lauric acid, myristic acid, palmitinic acid, stearic acid or mixtures thereof. Additionally preferred are organic polymers comprising acid groups.

Polymeric organic acids used preferably as component b) are homo- or copolymeric polycarboxylates or their partial neutralizates. Especially preferred are homopolymers or copolymers of acrylic acid and/or of methacrylic acid (referred to herein as “poly(meth)acrylic acid”), preferably in the form of their salts or in partially neutralized form. The used polymeric organic acid as a 1% solution in water should have a pH of 2 to 6.5, and preferably between 3.5 and 6.5. Homo- or copolymeric polycarboxylates, preferably with maleic acid, are concerned here. Polyacrylic acid or polymethacrylic acid for example are preferably used, especially those having an average molar mass of 500 to 70 000 g/mol.

Preference thereamong is given to poly(meth)acrylates, which preferably have a molar mass from 2.000 to 20.000 g/mol. Of this group, it is especially the short-chain poly(meth)acrylates with an average molar mass from 2.000 to 10.000 g/mol and preferably from 3.000 to 5.000 g/mol which are once again preferable by virtue of their superior solubility.

Preference thereamong is further given to copolymeric polycarboxylates, in particular those of acrylic acid with methacrylic acid and of acrylic acid or methacrylic acid with maleic acid. Copolymers of acrylic or methacrylic acid with maleic acid where the acrylic or methacrylic acid content is from 50 to 90 wt % and the maleic acid content is from 50 to 10 wt % have been found to be particularly suitable. Their average molar mass, referring to the free acid, is preferably in the range from 2.000 to 70.000 g/mol, more preferably in the range from 20.000 to 50.000 g/mol and most preferably in the range from 30.000 to 40.000 g/mol. Preferred polymers are Sokalan® CP45 and CP5 from BASF in substantially anhydrous form.

To improve their solubility in water, the polymers may also contain structural units derived from allylsulfonic acids, for example allyloxybenzenesulfonic acid and methallylsulfonic acid. Particular preference is also given to biodegradable polymers formed from more than two different monomer units, for example those containing structural units derived from salts of acrylic and/or methacrylic acid and maleic acid and also from vinyl alcohol and/or vinyl alcohol derivatives and sugar derivatives, or structural units derived from salts of acrylic and/or methacrylic acid and 2-alkylallylsulfonic acid and from sugar derivatives.

Preferred copolymers further include those containing structural units derived from acrolein and acrylic acid/acrylic acid salts or methacrylic acid/methacrylic acid salts, and/or acrolein and vinyl acetate.

Anionic polymers employed with preference as component b) further include sulfonated polymers, in particular copolymers formed from unsaturated carboxylic acids, sulfonated monomers with or without further ionogenic or nonionogenic monomers.

Especially preferred organic acids are citric acid, ascorbic acid and oxalic acid.

The performance of bleaching agents in washing and cleaning agents can be improved considerably when the per-compound is contacted with a combination of bleach catalyst and bleach activator. The bleaching effect of the catalyst is effectively supported by the peroxycarboxylic acid formed from the activator. At the same time, the peroxycarboxylic acid significantly contributes to the germ killing on the products to be cleaned, improves the odor of the washing liquor and prevents the formation of a biofilm in the washing machine or dishwasher. The combination of bleach catalysts and bleach activators is therefore useful for increasing the bleaching effect and for ensuring hygiene in the treatment of bleaching agents in washing agents and detergents.

The use of bleaching activators and bleach catalysts as separate particles or granulates, however, also includes disadvantages that can have negative effects on the bleaching effect. The reactions of the per-compound or the hydrogen peroxide released therefrom with the bleach activator and the bleach catalyst run in parallel. If the bleach catalyst granulate dissolves faster than the bleach activator granulates, then the per-compound is already consumed before it can react with the bleach activator. The same applies to the reverse case. Co-granulates from bleach activators and bleach catalysts are still advantageous in order to ensure the homogeneous distribution of both components in the detergent and cleaning agent and to save space in the formulation. Furthermore, the production costs are lowered, since in place of two different granulates only one co-granulate has to be produced.

Therefore, the invention preferably also relates to granulates comprising besides components a) and b) described above a bleach activator as component c).

Bleach activators that can be used in the granulates of the invention are, for example, polyacylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyloxy- or n-lauroyloxybenzenesulfonate (NOBS or LOBS), acylated phenolcarboxylic acids, in particular nonanoyloxy- or decanoyloxybenzoic acid (NOBA or DOBA), carboxylic anhydrides, in particular phthalic anhydride, acylated polyhydric alcohols, in particular triacetin, ethylene glycol diacetate and 2,5-diacetoxy-2,5-dihydrofuran, and acylated sorbitol and mannitol, or mixtures thereof (SORMAN), acylated sugar derivatives, in particular pentaacetylglucose (PAG), pentaacetylfructose, tetraacetylxylose and octaacetyllactose, and acetylated, optionally N-alkylated glucamine and gluconolactone, and/or N-acylated lactams, for example N-benzoylcaprolactam. Hydrophilically substituted acylacetals and acyllactams are likewise preferably used. In addition, nitrile derivatives such as n-methylmorpholinium acetonitrile methylsulfate (MMA) or cyanomorpholine (MOR) can be used as bleach activators. Combinations of conventional bleach activators can also be used.

Particularly preferred bleach activators are TAED, NOBS and DOBA.

As a further ingredient the granulates of the invention preferably contain a non-acidic binder d), in order to improve the cohesion of the granulates.

In another preferred embodiment, the invention relates to granulates containing besides the above described components a) and b) a non-acidic binder as component d).

In this embodiment, the granulates according to the invention can therefore be combinations of the components a), b) and d) or of the components a), b), c) and d).

Some of the acidic compounds described as component b), in particular polymeric organic acids, can also serve as a binder and also have a binder function in addition to the acid function. These compounds are counted as component b) within the scope of this description. The other binders, thus the non-acidic binders or the binders without acidic groups in the molecule, are counted as component d) within the scope of this description.

As binder d) preferably substances are used which are selected from alcohol alkoxylates and from polymers without acidic groups. Among the polymers without acidic groups, synthetic and natural polymers are understood as well as modified polymers of natural origin, which have no acidic groups, such as carboxylic groups or sulfonate groups.

Non-ionic polymers used with special preference as binder d) include polyvinyl alcohols, acetalisated polyvinyl alcohols, polyvinyl-pyrrolidones and polyalkylene glycols, in particular polyethylenoxide. Preferred polyvinyl alcohols and acetalisated polyvinyl alcohols have molecular weights in the range from 10,000 to 100,000 g/mol, preferably from 11,000 to 90,000 g/mol, particularly preferred from 12000 to 80,000 g/mol and especially preferably from 13,000 to 70,000 g/mol. Preferred polyethylenoxides have molar masses in the range of approximately 200 to 5,000,000 g/mol, corresponding to degrees of polymerization n from about 5 to >100,000.

Other preferred copolymers used as binders d) are those that have structural units derived from acrolein and vinyl acetate.

In another preferred embodiment, the invention relates to granulates containing besides the above described components a) and b) additional adjuvants as component e).

In this embodiment, the granulates according to the invention can therefore comprise combinations of the components a), b) and e) or of the components a), b), c) and e) or of the components a), b), d) and e) or of the components a), b), c), d) and e).

As additional adjuvants e) siccatives, such as calcium sulfate, come into consideration.

In the most simple embodiment of the invention, the granulates of the invention do not contain a sheath (so-called coating or protective layer).

In a preferred embodiment of the invention, the granulates of the invention are additionally provided with a sheath f), whereby the storage stability is further improved and the granulates can be dyed if necessary. The proportion of the sheath at the total quantity of the granulates can vary in wide ranges, but should be not more than 30 wt. %.

Preferred as a sheath are organic compounds having film-forming properties which are solid at 25° C., e.g. waxes, polyvinyl alcohols, methyl cellulose, carboxymethyl cellulose, hydroxymethylpropyl cellulose, hydroxymethyl cellulose, hydroxypropyl cellulose or the substances already used as film-forming acids b) and/or as binder d). Optionally, the sheath may contain additional small amounts of water-soluble or water-insoluble organic dyes.

In another preferred embodiment the granulate of the invention is equipped with two or more protective layers.

In a very preferred embodiment the granulate of the present invention comprises a first protective layer, preferably made from hydroxypropylmethyl cellulose (HPMC) and/or from methylcellulose (MC), and a second protective layer, preferably made from a fatty acid or a fatty acid mixture, most preferred from stearic acid and palmitic acid.

The quantity of component a) in the inventive granulates is usually from 0.1 to 30 wt. %, relative to the total amount of granulates. Preferred quantities of component a) are in the range from 2 to 12 wt. %.

The quantity of component b) in the inventive granulates is usually from 0.1 to 50 wt. %, relative to the total amount of granulates. Preferred quantities of component b) are in the range from 0.2 to 5 wt. % when non-film-forming compounds are concerned and in the range from 5 to 30 wt. % when film-forming compounds are concerned.

The quantity of component c) in the inventive granulates is usually from 0 to 80 wt. %, relative to the total amount of granulates. Preferred quantities of component c) are in the range from 40 to 80 wt. %, most preferred from 60 to 60% wt. %.

The quantity of component d) in the inventive granulates is usually from 0 to 80 wt. %, relative to the total amount of granulates. Preferred quantities of component d) are in the range from 15 to 75 wt. %, most preferred from 20 to 55 wt. %.

The quantity of component e) in the inventive granulates is usually from 0 to 25 wt. %, preferably from 0.1 to 25 wt. %, relative to the total amount of granulates. Most preferred quantities of component e) are in the range from 2 to 20 wt. %.

The quantity of component f) in the inventive granulates is usually from 0 to 30 wt. %, relative to the total amount of granulates. Preferred quantities of component f) are in the range from 0.5 to 10 wt. %.

Granulates of the present invention are particularly preferable with regard to their performance and storage stability when they contain, relative to the overall weight of the granulate,
a) 2 to 12% by weight of one or more bleach catalysts of formula IV, V or VII,
b) 0.2 to 5% by weight of citric acid, ascorbic acid and/or oxalic acid,
d) 20 to 55% by weight of one or more non-acid binders, and
e) 2 to 20% by weight of siccatives.

Granulates of the present invention are particularly preferable with regard to their performance and storage stability when they contain, relative to the overall weight of the granulate,
a) 2 to 12% by weight of one or more sulfonimines or one or more quaternary iminium salts,
b) 0.2 to 5% by weight of one or more organic acids which are solid at 25° C., and
c) 60 to 80% by weight of one or more bleach activators.

Preferably the granulates of the invention contain, relative to their overall weight,
a) 2 to 12% by weight of one or more bleach catalysts of formulae IV, V or VII,
b) 0.2 to 5% by weight of citric acid, ascorbic acid or oxalic acid,
c) 40 to 80% by weight of one or more bleach activators selected from the group of TAED, NOBS or DOBA,
d) 15 to 55% by weight of one or more non-acid binders, and
e) 2 to 20% by weight sikkatives.

Likewise preferably preferred the granulates of the invention contain, relative to their overall weight,
a) 2 to 8% by weight of one or more bleach catalysts of formulae IV, V or VII,
b) 5 to 30% by weight of one or more poly(meth)acrylic acids or their salts with a pH-value in the range from 3 to 8, and
c) 60 to 80% by weight of one or more bleach activators selected from the group TAED, NOBS or DOBA.

The above mentioned preferred granulates may have no coating f) or preferably have a coating f), in an amount from 0.5 to 10% by weight, relative to the overall weight of the granulate.

Various methods of granulation are possible in principle for providing the granulates of the present invention.

In a preferred manufacturing process the pulverulent actives first being mixed and the mixture then being compacted, thereafter ground with or without subsequent sieving into individual particle size fractions. The compacting is preferably effected on so-called roll compactors (e.g., from Hosokawa-Bepex, Alexanderwerk, Köppern). The roll profile can be varied to produce either pellets or briquettes or a compacted sheet. While it typically merely remains to separate the piece compacts from the fines, the sheet compact has to be comminuted in a mill down to the desired size of particle. Typically, the types of mill used are preferably of the gentle type, for example sieve and hammer mills (e.g., from Hosokawa-Alpine, Hosokawa-Bepex) or roll stands (e.g., from Bauermeister, Bühler).

The granulate material thus obtained is sieved to remove the undersize fraction and optionally the oversize fraction. The oversize fraction is preferably recycled to the mill, whereas the undersize fraction is recycled into the compaction process. The granules can be classified using commonplace sieving machines such as, for example, tumble or vibration sieves (e.g., from Allgaier, Sweco, Vibra).

Another preferred version of the manufacturing process of the granulates of the invention relates to mixing and ganulating of the ingredients in an intensive mixer. The granulate material thus obtained may be sieved to remove the undersize fraction and optionally the oversize fraction.

The granulates thus obtained may be provided with a coating in a separate step, for example by using a fluidised bed mixer.

The granulates of the present invention are primarily characterized by their chemical composition. It has nonetheless been found that the effect of these granules can also be advantageously influenced by influencing physical parameters such as, for example, the corpuscle size, the fines fraction and also the bleach catalyst content of selected sieved fractions.

Preferred granulates of the present invention are accordingly characterized in that they have an average corpuscle size between 0.1 and 1.6 mm, preferably between 0.2 and 1.2 mm and more preferably between 0.3 and 1.0 mm, as measured by sieve analysis.

In especially preferred granulates of the invention the mean volume size of the primary particles is in the range from 1 μm to 150 μm, and the finished granulates have an average particle size between 0.1 and 1.6 mm.

Preferred granulates of the invention are in addition characterized by a water content below 5% by weight (determined according to Karl Fischer), referring to the total amount of granulate.

Very preferred granulates of the invention have a water content of below 3% by weight, especially preferred between 0 and 2% by weight, referring to the total amount of the granulate.

The granulates of the present invention are suitable for use in any washing or cleaning compositions, although their use in dishwasher detergents, especially in machine dishwasher detergents has been found to be particularly advantageous.

It is believed that the acid in the granulates of the present invention performs a protective function and prevents the reaction of alkaline constituents of the washing composition with the non-alkali-resistant and hydrolysis-sensitive bleach catalysts and bleach activators also present in the granulates according to the present invention.

Another object of the present invention accordingly is the use of the granulates of the present invention for the preparation of washing agents and cleaning agents, preferably of dishwashing agents.

The present invention further also provides washing and cleaning compositions, preferably dishwasher detergents, comprising a granulate of the present invention.

Washing and cleaning agents containing the components a), b) and c) can be advantageously used at low temperatures and show excellent washing and cleaning results at temperatures of less than 35° C., preferably of less than 30° C. and especially preferably of less than 25° C. In addition, these washing and cleaning agents show an excellent germ-killing effect. The number of bacteria and yeasts on the treated substrates is significantly reduced by these agents.

Preferred washing and cleaning compositions according to the present invention, in particular the dishwasher detergents, incorporate the granulates of the present invention in amounts between 0.1 and 10 wt %, preferably in amounts between 0.2 and 8 wt % and more preferably in amounts between 0.5 and 6 wt %.

The washing and cleaning compositions of the present invention, in particular the dishwasher detergents, which may be in the form of granulates or in the form of pulverulent or tablet-shaped solids, but also in liquid or pasty form, may incorporate in principle any known and customary (in such compositions) ingredients in addition to the granulate of the present invention. The washing and cleaning compositions of the present invention, in particular the dishwasher detergents, may more particularly incorporate builder substances, peroxygen compounds, enzymes, alkali carriers, surfactants, pH regulators, organic solvents and further, auxiliary materials, such as glass corrosion inhibitors, silver corrosion inhibitors and foam regulators. The granulates of the present invention are useful in both phosphate-containing and phosphate-free formulations.

Particularly preferred washing and cleaning compositions, in particular dishwasher detergents, incorporate
i) 15 to 65 wt %, preferably 20 to 60 wt % of a water-soluble builder component,
ii) 5 to 25 wt %, preferably 8 to 17 wt %, of a peroxygen compound,
iii) 0.5 to 6 wt % of a granulate of the invention, and
iv) 0 to 50 wt % of further components such as enzymes, alkali carriers, surface active agents, pH regulators, organic solvents, glass corrosion inhibitors, silver corrosion inhibitors and foam regulators, all relative to the overall weight of the washing and cleaning composition.

A composition of this type is specifically of low alkalinity, i.e., its 1 weight percent solution has a pH in the range of 8 to 11.5, preferably of 9 to 11.

Water-soluble builder components for use in the washing and cleaning compositions of the present invention, in particular the dishwasher detergents, include in principle any builders typically used in such compositions. Examples thereof are alkali metal phosphates, which may be in the form of their alkaline, neutral or acidic sodium or potassium salts, preferably trisodium phosphate, tetrasodium diphosphate, disodium dihydrogendiphosphate, pentasodium triphosphate, so-called sodium hexametaphosphate and also the corresponding potassium salts and/or mixtures of sodium and potassium salts. Their amounts may be in the range from 15 to about 60 wt %, preferably from 20 to 60 wt %, relative to the overall composition. Further possible water-soluble builder components further include not only polyphosphonates and phosphonatoalkyl carboxylates but also, for example, organic polycarboxylate polymers of natural or synthetic origin which act as co-builders in hard-water regions in particular. Possibilities include, for example, polyacrylic acids and copolymers formed from maleic anhydride and acrylic acid, and also the sodium salts of these polymeric acids. Commercially available products include, for example, Sokalan™ CP 5, CP 10 and PA 30 from BASF. Useful co-builder polymers of natural origin include, for example, oxidized starch and polyamino acids such as polyglutamic acid or polyaspartic acid. Possible water-soluble builder components further include naturally occurring hydroxy carboxylic acids such as, for example, monohydroxysuccinic acid, dihydroxysuccinic acid, alpha-hydroxypropionic acid and gluconic acid. The preferred organic water-soluble builder components include the salts of citric acid, in particular sodium citrate.

Sodium citrate is anhydrous trisodium citrate and preferably trisodium citrate dihydrate. Trisodium citrate dihydrate is employable as finely or coarsely crystalline powder. Depending on the ultimate pH established in the washing and cleaning compositions of the present invention, in particular the dishwasher detergents, acids corresponding to the co-builder salts referred to may also be present. Particularly preferred builder components in phosphate-free formulations include methylglycine diacetate (MDGA, e.g., Trilon® M, BASF), L-glutamic acid, N,N-(biscarboxymethyl), tetrasodium salt (GLDA, Dissolvine® DL, Akzo Nobel), sodium polyaspartates (Baypure®, Lanxess) or salts of iminodisuccinic acid (Baypure®, Lanxess).

Preferred peroxygen compounds for use in the washing and cleaning compositions of the present invention, in particular the dishwasher detergents, include in principle any are perborates and percarbonates, in particular the corresponding sodium salts thereof.

The enzymes optionally incorporated in the washing and cleaning compositions of the present invention, in particular the dishwasher detergents, include proteases, amylases, pullulanases, cutinases and/or lipases, for example proteases such as BLAP™, Optimase™, Opticlean™, Maxacal™, Maxapem™, Durazym™, Purafect™ OxP, Esperase™ and/or Savinase™, amylases such as Termamyl™, Amylase-LT™, Maxamyl™, Duramyl™ and/or lipases such as Lipolase™, Lipomax™, Lumafast™ and/or Lipozym™. The enzymes used may be in a state of adsorption on carrier materials and/or embedment in enveloping substances in order that they may be protected against premature deactivation. They are incorporated in the washing and cleaning compositions of the present invention, in particular the dishwasher detergents, at preferably up to 10 wt % and more preferably at from 0.05 to 5 wt %, with particular preference in the form of enzymes stabilized against oxidative degradation.

The washing and cleaning compositions of the present invention, in particular the dishwasher detergents, preferably incorporate the usual alkali carriers such as, for example, alkali metal silicates, alkali metal carbonates and/or alkali metal bicarbonates. The alkali carriers typically used include carbonates, bicarbonates, and alkali metal silicates having an SiO₂/M₂O (M=alkali metal atom) molar ratio of 1:1 to 2.5:1. The alkali metal silicates may be incorporated at up to 40 wt %, in particular at from 3 to 30 wt %, relative to the overall composition. The alkali carrier system whose use in the washing and cleaning compositions of the present invention, in particular the dishwasher detergents, is preferred as a mixture of carbonate and bicarbonate, preferably sodium carbonate and sodium bicarbonate, and said mixture may be incorporated at up to 50 wt % and preferably at from 5 to 40 wt %.

In a further preferred embodiment of the invention, the washing and cleaning compositions of the invention, in particular the dishwasher detergents, incorporate from 20 to 60 wt % of water-soluble organic builders, in particular alkali metal citrate, from 3 to 20 wt % of alkali metal carbonate and from 3 to 40 wt % of alkali metal disilicate.

The washing and cleaning compositions of the present invention, in particular the dishwasher detergents, may optionally also incorporate surfactants, in particular anionic surfactants, zwitterionic surfactants and preferably low-sudsing nonionic surfactants, added for better detachment of greasy stains, as wetting agents and possibly as granulation assistants in the manufacture of these compositions. Their amount may be up to 20 wt %, preferably up to 10 wt %, and more preferably is in the range from 0.5 to 5 wt %, in each case referring to the total amount of the washing and cleaning composition.

Dishwasher detergents in particular typically utilize extremely low-foam compounds. These preferably include C₁₂-C₁₈ alkyl polyethylene glycol polypropylene glycol ethers each containing up to 8 mol of ethylene oxide and propylene oxide units in the molecule. However, it is also possible to use other renowned low-foam nonionic surfactants, for example C₁₂-C₁₈ alkyl polyethylene glycol polybutylene glycol ethers each containing up to 8 mol of ethylene oxide and butylene oxide units in the molecule, end capped alkyl polyalkylene glycol mixed ethers and also the admittedly sudsing, but ecologically attractive C₈-C₁₄ alkylpolyglucosides having a degree of polymerization of about 1 to 4 and/or C₁₂-C₁₄ alkyl polyethylene glycols having 3 to 8 ethylene oxide units in the molecule. Suitable are likewise surfactants from the family of glucamides such as, for example, alkyl-N-methylglucamides in which the alkyl moiety preferably derives from a fatty alcohol having a carbon chain length of C₆-C₁₄. It is advantageous in some instances when the surfactants described are employed in the form of mixtures, for example the combination of alkylpolyglycoside with fatty alcohol ethoxylates or glucamide with alkylpolyglycosides. The presence of amine oxides, betaines and ethoxylated alkylamines is also possible.

To establish a desired pH when not automatically resulting from mixing the other components, the washing and cleaning compositions of the present invention, in particular the dishwasher detergents, may incorporate system-compatible and environmentally compatible acids, in particular citric acid, acetic acid, tartaric acid, malic acid, lactic acid, glycolic acid, succinic acid, glutaric acid and/or adipic acid, but also mineral acids, in particular sulfuric acid or alkali metal hydrogensulfates, or bases, in particular ammonium hydroxide or alkali metal hydroxides. The level of pH regulators of this type in the washing and cleaning compositions of the present invention, in particular the dishwasher detergents, preferably does not exceed 10 wt % and more preferably is in the range from 0.5 to 6 wt %, in each case referring to the total amount of the washing and cleaning composition.

Organic solvents useful in the washing and cleaning compositions of the present invention, in particular the dishwasher detergents, in particular when these are in liquid or pasty form, include alcohols of 1 to 4 carbon atoms, in particular methanol, ethanol, isopropanol and tert-butanol, diols of 2 to 4 carbon atoms, in particular ethylene glycol and propylene glycol, and also mixtures thereof, and the ethers derivable from the recited classes of compounds. The level of such water-miscible solvents in the washing and cleaning compositions of the present invention, in particular dishwasher detergents, preferably does not exceed 20 wt % and more preferably is in the range from 1 to 15 wt %.

To inhibit glass corrosion during the wash cycle, the washing and cleaning compositions of the present invention, in particular the dishwasher detergents, may incorporate glass corrosion inhibitors. Crystalline layered silicates and/or zinc salts are particularly advantageous here. Crystalline layered silicates are available for example from Clariant under the trade name of Na-SKS, e.g. Na-SKS-1 (Na₂Si₂₂O₄₅.xH₂O, kenyaite), Na-SKS-2 (Na₂Si₁₄O₂₉.xH₂O, magadiite), Na-SKS-3 (Na₂Si₈O₁₇.xH₂O) or Na-SKS-4 (Na₂Si₄O₉.xH₂O, makatite). Suitable among these are in particular Na-SKS-5 (alpha-Na₂Si₂O₅), Na-SKS-7 (beta-Na₂Si₂O₅, natrosilite), Na-SKS-9 (NaHSi₂O₅.H₂O), Na-SKS-10 (NaHSi₂O₅.3H₂O, kanemite), Na-SKS-11 (t-Na₂Si₂O₅) and Na-SKS-13 (NaHSi₂O₅), but in particular Na-SKS-6 (delta-Na₂Si₂O₅). An overview of crystalline sheet-silicates is found, for example, in the article published in “Seifen-Öle-Fette-Wachse, 116 volume, No. 20/1990”, on pages 805-808.

In a further preferred embodiment, the washing and cleaning compositions of the present invention, in particular the dishwasher detergents, incorporate the crystalline layered silicate at preferably 0.1 to 20 wt %, more preferably 0.2 to 15 wt % and more preferably 0.4 to 10 wt %, all relative to the overall weight of the composition.

To control glass corrosion, washing and cleaning compositions of the present invention, in particular dishwasher detergents, may incorporate at least one zinc or bismuth salt, preferably selected from the group of organozinc salts, more preferably selected from the group of soluble organozinc salts, yet more preferably selected from the group of soluble zinc salts of monomeric or polymeric organic acids and yet still more preferably selected from the group consisting of zinc acetate, zinc acetylacetonate, zinc benzoate, zinc formate, zinc lactate, zinc gluconate, zinc oxalate, zinc ricinoleate, zinc abietate, zinc valerate and zinc p-toluenesulfonate. Bismuth salts such as, for example, bismuth acetates are employable as an alternative to or in combination with these zinc salts.

Preference in the context of the present invention is given here to washing and cleaning compositions, in particular dishwasher detergents, where the amount of zinc salt, relative to the overall weight of this composition, is from 0.1 to 10 wt %, preferably from 0.2 to 7 wt % and more preferably from 0.4 to 4 wt %, irrespective of which zinc salts are used, specifically irrespective that is as to whether organic or inorganic zinc salts, soluble or insoluble zinc salts or mixtures thereof are used.

Silver corrosion inhibitors may be incorporated in the washing and cleaning compositions, in particular dishwasher detergents, of the invention for silver corrosion control. Preferred silver corrosion inhibitors are organic sulfides such as cystine and cysteine, di- or trihydric phenols, optionally alkyl- or aryl-substituted triazoles such as benzotriazole, isocyanuric acid, salts and/or complexes of titanium, of zirconium, of hafnium, of cobalt or of cerium wherein the metals referred to are present in one of the oxidation states II, III, IV, V or VI, depending on the metal.

When the washing and cleaning compositions of the invention, in particular the dishwasher detergents, are prone to excessive sudsing in use in the presence of anionic surfactants, for example, they may be additionally additized with up to 6 wt %, preferably about 0.5 to 4 wt %, of a foam-suppressing compound, preferably from the group of silicone oils, mixtures of silicone oil and hydrophobicized silica, paraffins, paraffin-alcohol combinations, hydrophobicized silica, bisfatty acid amides, and other further known commercially available defoamers.

Further possible ingredients for the washing and cleaning compositions of the present invention, in particular the dishwasher detergents, include, for example, the sequestrants, electrolytes, additional peroxygen activators, dyes or fragrances such as, for example, perfume oils that are familiar from the prior art relating to such compositions.

The production of solid washing and cleaning compositions of the present invention, in particular the dishwasher detergents, does not present any difficulties and may in principle be carried out in a known manner, for example by spray-drying or granulation, in which case the peroxygen compound and the granulate material of the present invention are optionally added separately at a later stage.

A particularly advantageous way to produce those washing and cleaning compositions of the present invention which take the form of aqueous solutions or of solutions containing other customary solvents, in particular such dishwasher detergents, is to simply mix the ingredients, which may be introduced into an automatic mixer in the form of a solution or without a solvent.

The washing and cleaning compositions, in particular dishwasher detergents, of the present invention preferably take the form of pulverulent, granular or tableted preparations obtainable in a known manner, for example by mixing, granulating, roll compacting and/or spray drying the thermally robust components and subsequently admixing the more sensitive components, which must be considered to include specifically enzymes, bleaching agents and the bleach catalyst.

To produce washing and cleaning compositions, in particular dishwasher detergents, of the present invention in tablet form, a preferable procedure involves mixing all the constituents with one another in a mixer and using conventional tableting presses, for example eccentric presses or rotary presses, to mold the mixture using molding pressures in the range from 200×10⁵ Pa to 1500×10⁵ Pa.

The tablets thus obtained in a straightforward manner, which normally have flexural strengths of above 150 N, are fracture-resistant but will nonetheless dissolve sufficiently rapidly under the intended conditions of service. A tablet thus obtained preferably combines a weight of 15 to 40 g, in particular 20 to 30 g, with a diameter of 35 to 40 mm.

Those washing and cleaning compositions of the present invention which take the form of nondusting, storage-stably free-flowing powders and/or granulates having high bulk densities in the range from 800 to 1000 g/I, in particular such dishwasher detergents of the present invention, are obtainable in a process wherein, in a first subsidiary step of the process, the builder components are mixed with at least a proportion of liquid mixing components to increase the bulk density of this pre-mix and then—if desired after an intervening drying operation—the further constituents of the composition, including the bleach catalyst granulate of the present invention, are combined with the pre-mix thus obtained.

Dishwasher detergents of the present invention are useful not only in domestic dishwashers but also in industrial/institutional dishwashers. They are added by hand or by means of suitable dosing devices. The use concentrations in the cleaning liquor are generally about 1 to 8 g/l, preferably 2 to 5 g/l. The same applies to the use of the inventive washing and cleaning agents.

In a dishwasher generally, the actual wash cycle is advantageously followed by several intermediate rinse cycles with clear water and a final rinse cycle with a customary rinse aid. The dishes obtained after drying are spotlessly clean and hygienically impeccable when dishwasher detergents according to the present invention are used.

### EXAMPLES

In the examples which follow, % ages are weight percent (wt %) unless specifically stated otherwise. As regards the reported relative humidities, the % ages have the usual meaning.

### Examples 1-3: Preparation of Granulates According to the Invention

The single components of the compositions of the invention were mixed and granulated in an Eyrich intensive mixer. The amounts and ingredients of single examples are reported in table 1 hereinbelow.

TABLE 1
Composition of the granulates of Examples 1-3

| | Example no. | | |
|---|---|---|---|
| | 1 | 2 | 3 |
| 3-methyl-1,2-benzisothiazole-1,1- | 9.8 | 9.1 | 9.1 |
| dioxide (%) | | | |
| microcrystalline cellulose (%)¹⁾ | 19.8 | 18.4 | 18.4 |
| corn starch (%) | | 28.1 | 28.1 |
| rice starch (%) | 30.3 | | |
| microcrystalline cellulose (%)²⁾ | 10.9 | | 10.1 |
| microcrystalline cellulose (%)³⁾ | | 10.1 | |
| calcium sulfate (%) | 17.5 | 16.2 | 16.2 |
| polyvinylalcohol (%)⁴⁾ | 9.1 | 8.5 | 8.5 |
| citric acid (%) | 0.3 | 0.3 | 0.3 |
| disodiumhydrogencitrate (%) | 0.3 | 0.3 | 0.3 |

¹⁾Arbocel ® FDY 660 from JRS
²⁾Vivapur ® 200 from JRS
³⁾Heweten ® 101 from JRS
⁴⁾Poval ® 6-88 from Kuraray

The granulates prepared in this manner were subsequently provided with a coating in a fluidized-bed coating apparatus from Fa. Glatt.

The granulate of Example 1 obtained a coating of 2.2% by weight of hydroxy-methylcellulose.

The granulate of Example 2 obtained a coating of 9.1% by weight of hydroxy-methylcellulose.

The granulate of Example 3 obtained a coating of 9.1% by weight of hydroxypropyl-cellulose.

### Application Examples

To test the chemical and physical stability of the granulates of Examples 1 to 3, their storage behavior was investigated in a typical laundry detergent powder formulation. To this end, the granulates of Examples 1 to 3 were incorporated in IEC-A base laundry detergent powder such that the final formulation contained 3% of granulate. The mixture was transferred into a glass bottle and stored for several days with a closed lid under climatic conditions (T=32° C., 75% relative humidity). The parameter evaluated was the color of the granulate in the laundry detergent powder mixture after 14 days. As a comparative instead of the granulate pulvurulent 3-methyl-1,2-benzisothiazole-1,1-dioxide was incorporated into the IEC-A base laundry detergent powder. The results are shown in table 2 hereinbelow

TABLE 2
Results of storage tests

| Granulate | Color | Color (14 days) |
|---|---|---|
| Example 1 | slightly yellowish | slightly yellowish |
| Example 2 | slightly yellowish | slightly yellowish |
| Example 3 | slightly yellowish | slightly yellowish |
| pulvurulent 3-methyl-1,2- | slightly yellowish | yellow-orange |
| benzisothiazole-1,1-dioxide | | |

1. Granulate comprising
a) one or more sulfonimines and/or one or more quaternary iminium salts, and
b) one or more acids which are solid at 25° C.

2. Granulate according to claim 1, wherein the sulfonimine is a compound of formula I 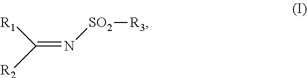
in which R₁ is hydrogen, alkyl, cycloalkyl, aryl, aralkyl or heterocyclyl, in which the residues alkyl, cycloalkyl, aryl, aralkyl or heterocyclyl are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups,
R₂ is hydrogen, alkyl, alkoxy, cycloalkyl, aryl, aralkyl, heterocyclyl, nitro, cyano, halogen, carboxyl, carboxylic ester or carboxylic amide, in which the residues alkyl, cycloalkyl, aryl, aralkyl or heterocyclyl are unsubstituted or have one to three substituents selected from the group alkyl, alkoxy, aryl, heterocyclyl, amino, hydroxy, carboxyl, carboxylic ester, carboxylic amide, sulfonyl, cyano or halogen, or in which the residues alkyl, cycloalkyl, aryl or heterocyclyl are substituted with a residue of formula —R₁C═N—SO₂—R₃ or in which an alkyl group is interrupted by one or more not directly adjacent oxygen atoms or by one or more not directly adjacent keto groups,
R₃ is alkyl, cycloalkyl, aryl, aralkyl, heterocyclyl, nitro, cyano or halogen, in which the residues alkyl, cycloalkyl, aryl, aralkyl or heterocyclyl are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups, or in which
R₁ and R₂ together with the carbon atom to which they are attached form a cycloaliphatic, heterocyclic or carbocyclic-aromatic ring system, which are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups, or in which
R₂ and R₃ together with the group —C═N—SO₂—, to which they are attached, form a heterocyclic ring system which is unsubstituiert or has one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups.

3. Granulate according to claim 2, wherein R₁ is hydrogen, R₂ is an unsubstituted phenyl or a phenyl which is substituted in 4-position with carboxyl, chlorine, cyano, hydroxy or methoxy, and R₃ is unsubstituted phenyl or phenyl which is substituted in 4-position with carboxyl, chlorine or nitro.

4. Granulate according to claim 1, wherein the sulfonimine is a compound of formula II, III, IV or V 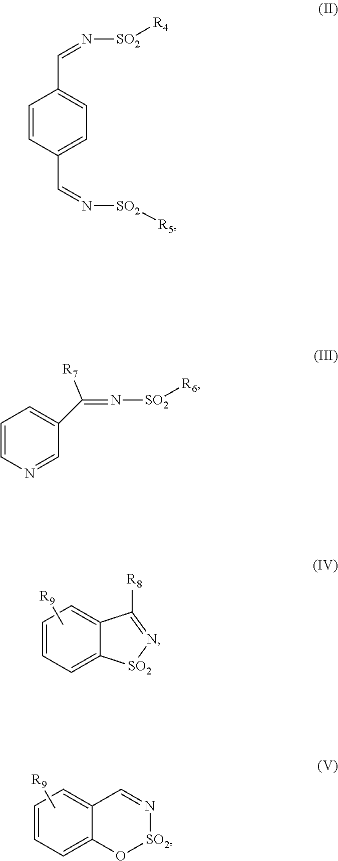
in which R₄ and R₅ independently of one another are unsubstituted phenyl or phenyl which is substituted in 4-position with carboxyl, chlorine, cyano, hydroxy or methoxy,
R₇ is hydrogen, C₁-C₆-alkyl or phenyl,
R₆ is unsubstituted phenyl or phenyl which is substituted in 4-position with carboxyl, chlorine, cyano, hydroxy or methoxy,
R₈ and R₉ independently of one another are hydrogen, C₁-C₆-alkyl or phenyl, and
R₉ is hydrogen, C₁-C₆-alkyl or phenyl.

5. Granulate according to claim 4, wherein the sulfonimine is a compound of formula IV, in which R₉ is hydrogen and R₅ is C₁-C₆-alkyl or phenyl.

6. Granulate according to claim 1, wherein the quaternary iminium salt is a compound of formula VI 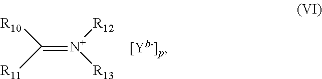
in which R₁₀ and R₁₂ independently of one another are hydrogen, alkyl, cycloalkyl, aryl, aralkyl or heterocyclyl, in which the residues alkyl, cycloalkyl, aryl, aralkyl or heterocyclyl are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups,
R₁₁ is hydrogen, alkyl, alkoxy, cycloalkyl, aryl, aralkyl, heterocyclyl, nitro, cyano, halogen, carboxyl, carboxylic ester or carboxylic amide, in which the residues alkyl, cycloalkyl, aryl, aralkyl or heterocyclyl are unsubstituted or have one to three substituents selected from the group alkyl, alkoxy, aryl, heterocyclyl, amino, hydroxy, carboxy, carboxylic ester, carboxylic amide, sulfonyl, cyano, hydroxy or halogen, or in which an alkyl group is interrupted by one or more not directly adjacent oxygen atoms or by one or more not directly adjacent keto groups,
R₁₃ is alkyl, cycloalkyl, aryl, aralkyl, heterocyclyl, nitro, cyano or halogen, in which the residues alkyl, cycloalkyl, aryl, aralkyl or heterocyclyl are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups, or in which
R₁₀ and R₁₂ together with the group —C═N⁺—, to which they are attached, form a heterocyclic ring system, which is unsubstituted or has one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more non directly adjacent oxygen atoms or by one or more not directly adjacent keto groups, or in which
R₁₁ and R₁₃ together with the group —C═N⁺—, to which they are attached, form a heterocyclic ring system, which is unsubstituted or has one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups,
Y is a b-valent inorganic or organic counterion,
b is an integer of at least 1, preferably from 1 to 3, more preferably 1 or 2 and most preferably 1, and
p is a rational number with value of 1/b.

7. Granulate according to claim 6, wherein the quaternary iminium salt is a compound of formula VII 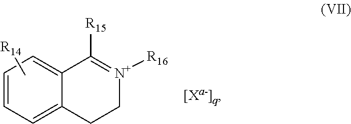
in which R₁₄ and R₁₅ independently of one another are hydrogen, alkyl, alkoxy, cycloalkyl, aryl, aralkyl, heterocyclyl, nitro, cyano, halogen, carboxyl, carboxylic ester or carboxylic amide, in which the residues alkyl, cycloalkyl, aryl, aralkyl or heterocyclyl are unsubstituted or have one to three substituents selected from the group alkyl, alkoxy, aryl, heterocyclyl, amino, hydroxy, carboxy, carboxylic ester, carbonxylic amide, sulfonyl, cyano, hydroxy or halogen, or in which an alkyl group is interrupted by one or more not directly adjacent oxygen atoms or by one or more not directly adjacent keto groups,
R₁₆ is alkyl, aryl, aralkyl or heterocyclyl, in which these residues are unsubstituted or have one to three substituents selected from the group alkyl, alkoxy, aryl, heterocyclyl, amino, carboxyl, carboxylic ester, carboxylic amide, sulfonyl, cyano, hydroxy or halogen, or in which an alkyl group is interrupted by one or more not directly adjacent oxygen atoms or by one or more not directly adjacent keto groups,
X is an a-valent inorganic or organic anion, and
a is an integer of at least 1, preferably from 1 to 3, more preferably 1 or 2 and most preferably 1, and
q is a rational number with value of 1/a.

8. Granulate according to claim 7, wherein R₁₆ is C₁-C₁₅-alkyl or phenyl, benzyl, 2-hydroxyethyl, dimethylaminoethyl, ethoxyethyl or 2-pyridyl, R₁₄ is hydrogen, C₁-C₁₅-alkyl, trifluoromethyl, methoxy, nitro, fluorine, chlorine, bromine, cyano, phenyl, propyl-2-one, acetyl or pyridyl, R₁₅ is hydrogen, C₁-C₆-alkyl or phenyl, X is selected from the group chloride, bromide, tetrafluoroborate, hexafluorophosphate, methanesulfonate, ethanesulfonate or p-toluoenesulfonate, and a and q each are 1.

9. Granulate according to claim 1, wherein component b) is selected from the group of citric acid, ascorbic acid, oxalic acid, adipic acid, sebacic acid, malonic acid, succinic acid, glutaric acid, malic acid, tartaric acid, lactic acid, maleic acid, fumaric acid, saccharic acids, aminocarboxylic acids, C₈-C₂₂-fatty acids, a homo- or copolymeric polycarboxylate, the partial neutralisates thereof or mixtures of these.

10. Granulate according to claim 9, wherein component b) is selected from the group of oxalic acid, ascorbic acid, citric acid and/or the C₈-C₂₂-fatty acids.

11. Granulate according to claim 1, further comprising one or more bleach activators as component c).

12. Granulate according to claim 11, further comprising one or more non-acidic binders as component d).

13. Granulate according to claim 12, further comprising one or more additional additives as component e).

14. Granulate according to claim 13, further provided with a coating as component f).

15. Granulate according to claim 4, comprising ingredients in the following amounts, each referred to the total amount of the granulate
a) 2 to 12% by weight of one or more bleach catalysts of formulae IV or V,
b) 0.2 to 5% by weight of citric acid, ascorbic acid and/or oxalic acid,
c) 20 to 55% by weight of one or more non-acidic binders, and
d) 2 to 20% by weight of siccatives.

16. Granulate according to claim 7, comprising ingredients in the following amounts, each referred to the total amount of the granulate
a) 2 to 12% by weight of one or more bleach catalysts of formula VII,
b) 0.2 to 5% by weight of citric acid, ascorbic acid and/or oxalic acid,
c) 20 to 55% by weight of one or more non-acidic binders, and
d) 2 to 20% by weight of siccatives.

17. Granulate according to claim 4, comprising ingredients in the following amounts, each referred to the total amount of the granulate
a) 2 to 12% by weight of one or more sulfonimines or one or more quaternary iminium salts,
b) 0.2 to 5% by weight of one or more organic acids which are solid at 25° C., and
c) 60 to 80% by weight of one or more bleach activators.

18. Granulate according to claim 7, comprising ingredients in the following amounts, each referred to the total amount of the granulate
a) 2 to 12% by weight of one or more sulfonimines or one or more quaternary iminium salts,
b) 0.2 to 5% by weight of one or more organic acids which are solid at 25° C., and
c) 60 to 80% by weight of one or more bleach activators.

19. Granulate according to claim 4, comprising ingredients in the following amounts, each referred to the total amount of the granulate
a) 2 to 12% by weight of one or more bleach catalysts of formulae IV or V,
b) 0.2 to 5% by weight of citric acid, ascorbic acid or oxalic acid,
c) 40 to 80% by weight of one or more bleach activators selected from the group TAED, NOBS or DOBA,
d) 15 to 55% by weight of one or more non-acidic binders, and
e) 2 to 20% by weight of siccatives.

20. Granulate according to claim 7, comprising ingredients in the following amounts, each referred to the total amount of the granulate
a) 2 to 12% by weight of one or more bleach catalysts of formula VII,
b) 0.2 to 5% by weight of citric acid, ascorbic acid or oxalic acid,
c) 40 to 80% by weight of one or more bleach activators selected from the group TAED, NOBS or DOBA,
d) 15 to 55% by weight of one or more non-acidic binders, and
e) 2 to 20% by weight of siccatives.

21. Granulate according to claim 4, comprising ingredients in the following amounts, each referred to the total amount of the granulate
a) 2 to 8% by weight of one or more bleach catalysts of formulae IV or V,
b) 5 to 30% by weight of one or more poly(meth)acrylic acids or their salts with a pH-value in the range between 3 and 8, and
c) 60 to 80% by weight of one or more bleach activators selected from the group TAED, NOBS or DOBA.

22. Granulate according to claim 7, comprising ingredients in the following amounts, each referred to the total amount of the granulate
a) 2 to 8% by weight of one or more bleach catalysts of formula VII,
b) 5 to 30% by weight of one or more poly(meth)acrylic acids or their salts with a pH-value in the range between 3 and 8, and
c) 60 to 80% by weight of one or more bleach activators selected from the group TAED, NOBS or DOBA.

23. A method of making washing agents and cleaning agents comprising utilizing a granulate according to claim 1.

24. Washing agent and cleaning agent comprising a granulate according to claim 1.

25. Washing agent and cleaning agent according to claim 24, wherein the washing and cleaning agent is an agent for cleaning of dishes.

26. Washing agent and cleaning agent according to claim 24, wherein the granulate contains, in addition to sulfonimines and/or quaternary iminium salts as component a) and acids being solid at 25° C. as component b), further contains bleach activators as component c) and is suitable as a washing agent or cleaning agent at temperatures of less than 35° C.
